(19) United States
(12) Patent Application Publication
(10) Pub. No.: US 2026/0167929 A1
(43) Pub. Date: Jun. 18, 2026

(54) CULTURE MEDIUM AND CULTURE METHOD FOR INDUCING OLFACTORY MUCOSA MESENCHYMAL STEM CELLS INTO OLFACTORY ENSHEATHING CELLS

(71) Applicant: HUNAN BIZU BIOTECHNOLOGY CO., LTD., Changsha (CN)

(72) Inventors: Li WEN, Changsha (CN); Wenshui LI, Changsha (CN); Wen LU, Changsha (CN); Li HU, Changsha (CN); Yu LIU, Changsha (CN); Wei QIN, Changsha (CN)

(21) Appl. No.: 19/412,442

(22) Filed: Dec. 8, 2025

Related U.S. Application Data
(63) Continuation-in-part of application No. PCT/CN2025/120926, filed on Sep. 12, 2025.

(30) Foreign Application Priority Data
Dec. 16, 2024 (CN) 202411849756.3

(51) Int. Cl. C12N 5/079

(52) U.S. Cl. CPC C12N 5/0622; C12N 2500/05; C12N 2500/24; C12N 2501/13; C12N 2501/33; C12N 2501/39; C12N 2506/1392

(57) ABSTRACT

A culture medium and culture method for inducing olfactory mucosa mesenchymal stem cells into olfactory ensheathing cells are provided, belonging to the technical field of cell induction and culture. The culture medium provided in the present disclosure is mainly composed of cellular trophic factors, and does not contain animal proteins. The present disclosure further provides a method for inducing olfactory mucosa mesenchymal stem cells into olfactory ensheathing cells using the culture medium.

### CROSS REFERENCE TO RELATED APPLICATION

The application is a continuation-in-part of International Patent Application No. PCT/CN 2025/120926, filed on Sep. 12, 2025, which claims priority of the Chinese patent application with the application number “202411849756.3”, filed on Dec. 16, 2024, both of which are incorporated by references herein their entities for all purposes.

### TECHNICAL FIELD

The present disclosure belongs to the technical field of cell culture, and relates to a culture medium and culture method for inducing olfactory mucosa mesenchymal stem cells into olfactory ensheathing cells.

### BACKGROUND

Olfactory ensheathing cells (OECs) are a special type of glial cell functionally intermediate between Schwann cells and oligodendrocytes. OECs may not only secrete BDNF, NGF Neurotrophin-3 (NT3), and NGF neurotrophin-4 (NT-4), but also express fibronectin, laminin, L1, S100, glial-derived connexin, and neural cell adhesion molecules, all of which may support axonal extension.

Since OECs secrete neurotrophic factors and axonal growth stimulating substances that may facilitate regeneration of axons and the formation of myelin sheath, they have become one of the ideal candidate cells for promoting regeneration of central nervous. They are currently widely used in nervous system diseases, such as spinal cord injury, brain injury, and neurodegenerative diseases. Due to the high demand for olfactory ensheathing cells, current scientific research mainly relies on extracting olfactory ensheathing cells from the olfactory bulb tissue of animals, which is inefficient and clinically inapplicable. On the other hand, methods for directly obtaining and culturing olfactory ensheathing cells on the basis of human nasal mucosa tissue are complex, and the tissue source is not conducive to long-term supply. Therefore, there is an urgent need for an obtaining method with convenient and stable sourced and clinical application in the future.

The method for inducing the directional differentiation of olfactory mucosa mesenchymal stem cells into dopaminergic neurons of the present disclosure suggests that olfactory mucosa mesenchymal stem cells may be converted into nerve cells. Olfactory mucosa mesenchymal stem cells have a multipotent differentiation potential, and, like olfactory ensheathing cells, are derived from the ectoderm. The differentiation of olfactory mucosa mesenchymal stem cells into olfactory ensheathing cells does not require trans-germ layer conversion.

The source of olfactory mucosa mesenchymal stem cells does not require repeated obtaining, and a large number of olfactory mucosa mesenchymal stem cells may be obtained by a single extraction of nasal mucosa tissue. Thus, olfactory mucosa mesenchymal stem cells may serve as a stable source of olfactory ensheathing cells.

Therefore, there is an urgent need to provide a culture medium and culture method for inducing olfactory mucosa mesenchymal stem cells into olfactory ensheathing cells, providing a strong guarantee for their future clinical application.

### SUMMARY

In view of this, an objective of the present disclosure is to provide a culture medium and culture method for inducing olfactory mucosa mesenchymal stem cells into olfactory ensheathing cells.

The present disclosure provides a cell culture medium for inducing olfactory mucosa mesenchymal stem cells into olfactory ensheathing cells, where the cell culture medium includes DMEM/F12 as a basal medium, and further includes transferrin, insulin, hydrocortisone, sodium selenate, N-2 Supplement, and Neurotrophin-3(NT3).

The present disclosure further provides a method for inducing olfactory mucosa mesenchymal stem cells into olfactory ensheathing cells, including the steps of: culturing olfactory mucosa mesenchymal stem cells using the cell culture medium.

The present disclosure relates to an olfactory ensheathing cell solution prepared by the above method, where the olfactory ensheathing cells express olfactory ensheathing cell markers GFAP and S100β, with a purity of 65% or more.

### BRIEF DESCRIPTION OF THE DRAWINGS

FIG. 1 shows the morphology of induced olfactory ensheathing cells under light microscopy;

FIG. 2 shows the expression of olfactory ensheathing cell markers detected by immunofluorescence;

FIG. 3 shows the expression of olfactory ensheathing cell markers detected by flow cytometry.

### DETAILED DESCRIPTION OF THE EMBODIMENTS

The technical solutions of the present disclosure will be clearly and completely described below with reference to the examples of the present disclosure. Apparently, the examples described are merely some rather than all of the examples of the present disclosure. Based on the examples of the present disclosure, all other examples that would have been obtained by those of ordinary skill in the art without any creative efforts shall fall within the scope of protection of the present disclosure.

The present disclosure provides a cell culture medium for inducing olfactory mucosa mesenchymal stem cells into olfactory ensheathing cells, where the cell culture medium includes DMEM/F12 as a basal medium, and further includes transferrin, insulin, hydrocortisone, sodium selenate, N-2 Supplement, and NT3. DMEM/F12 is a commercial conventional culture medium, and the DMEM/F12 used in this article is Gibco brand, Lot/Batch number: 6125207; REF is C1330500BT. The specific components of DMEM/F12 are: DMEM, glucose, amino acids, vitamins, zinc, putrescine, hypoxanthine, thymidine, L-glutamine, phenol red indicator HEPES. N-2 Supplement is a commercial conventional cell culture additive, and the brand used in this article is Gibco, item number: 17502048. The specific components of N-2 Supplement are: human transferrin (Holo), recombinant full chain insulin, progesterone, putrescine, and transparent gypsum.

In an exemplary embodiment, the cell culture medium includes DMEM/F12 as a basal medium, and further includes 0.1 to 0.5 mg/L transferrin, 0.5 to 1.0 μL/L insulin, 0.5 to 1.0 μL/L hydrocortisone, 0.1 to 0.5 μg/ml sodium selenate, 0.1 to 0.5 mL/L N-2 Supplement, and 0.01 to 0.05 μg/L NT3.

In another exemplary embodiment, the cell culture medium includes DMEM/F12 as a basal medium, and further includes 0.2 mg/L transferrin, 0.5 μL/L insulin, 0.8 μL/L hydrocortisone, 0.4 μg/ml sodium selenate, 0.3 mL/L N-2 Supplement, and 0.03 μg/L NT3.

Basal medium DMEM/F12 is a widely used basal medium that provides rich nutrients and trace elements to support cell growth and differentiation, and may be used to sustain the growth of many types of mammalian cells. Transferrin is a protein that may bind to and transport iron ions. In the process of inducing the differentiation of olfactory mucosa mesenchymal stem cells, sufficient energy is required for the transformation of cell morphology and function. The iron ions transported by transferrin may maintain intracellular and extracellular iron balance, promote cell growth and proliferation, and avoid the formation of oxygen free radicals in the extracellular matrix. Additionally, transferrin may also participate in cellular signal transduction processes, indirectly modulating cell differentiation. Insulin plays an important role in cellular metabolism, which may promote cellular uptake of glucose and amino acids, lipogenesis, monovalent cation and phosphate transport, proteins and nucleic acid synthesis, and promote cell proliferation. For the induction of olfactory mucosa mesenchymal stem cells into olfactory ensheathing cells, insulin may promote the cellular uptake of nutrients such as glucose, providing a sufficient energy and material basis for cell differentiation. Moreover, insulin may also modulate cellular protein synthesis, aiding cells in constructing new protein structures, which is essential for morphological and functional transformation. Hydrocortisone, an adrenocortical hormone, has anti-inflammatory and immunomodulatory effects, helping maintain cellular stability and prolonging cell lifespan. During the induction of olfactory mucosa mesenchymal stem cells into olfactory ensheathing cells, hydrocortisone may modulate gene expression. It may also modulate the inflammatory response of cells and reduce interference from inflammation during cell culture, which is beneficial for stable cell differentiation. Sodium selenite contains selenium, a cofactor for glutathione peroxidase and several other important enzymes, which has an antioxidant effect, protecting cells from oxidative stress damage. N-2 Supplement is a cell culture additive that is mainly used for the culture of nerve cells. It contains a variety of components, such as saturated transferrin, recombinant insulin, and progesterone. These components may provide nutrients and signaling molecules needed for cell growth, promoting cell survival, proliferation and differentiation, thereby facilitating the conversion of olfactory mucosa mesenchymal stem cells into olfactory ensheathing cells. NT3 (Neurotrophin-3), a member of the neurotrophin family, is one of the important components in promoting nerve cell growth and development. During the induction of olfactory mucosa mesenchymal stem cells into olfactory ensheathing cells, NT3 may stimulate cell differentiation and promote the conversion toward olfactory ensheathing cells. It may activate a series of intracellular signaling pathways involved in regulating a variety of cellular processes, such as cell proliferation, survival, and differentiation, helping the better differentiation of olfactory mucosa mesenchymal stem cells into olfactory ensheathing cells. These components work together to provide a suitable growth and differentiation environment for olfactory mucosa mesenchymal stem cells, thereby inducing the differentiation of olfactory mucosa mesenchymal stem cells into olfactory ensheathing cells.

The present disclosure further provides a method for inducing olfactory mucosa mesenchymal stem cells into olfactory ensheathing cells, including the steps of: culturing olfactory mucosa mesenchymal stem cells using the cell culture medium.

In an exemplary embodiment, the method includes the steps of:
(1) culturing olfactory mucosa mesenchymal stem cells to passage 2 (P2);
(2) digesting the cells using trypsin, and performing centrifugation;
(3) removing the supernatant from the centrifuge tube, and collecting the pellet;
(4) uniformly mixing the cell pellet with the cell culture medium to obtain a mixed solution;
(5) uniformly inoculating the mixed solution into a cell plate, and placing the plate in an incubator for culture;
(6) after 1 day of culture, removing the supernatant and the floating cells, and replenishing the cell culture medium, followed by regular medium replacement; and performing continuous culture for 14 days to obtain the olfactory ensheathing cells.

In an embodiment, the cell plate is a 6-well plate, with an inoculating volume of 2 mL/well.

In an embodiment, in step (2), the concentration of the trypsin is 0.25 % (w/v%), the digesting time is 30 seconds, the rotation speed of the centrifugation is 1000 rpm, and the centrifugation time is 5 minutes.

In an embodiment, in step (4), the inoculating density of cells in the cell culture medium is 10⁵ cells/ml.

In an embodiment, in step (1), the olfactory mucosa mesenchymal stem cells are cultured using an olfactory mucosa mesenchymal stem cell culture medium consisting of DMEM/F12 and 10%(/v) bovine serum until reaching 70-80% cell confluence to obtain P2.

Compared with the conventional technology, the present disclosure has the following beneficial effects:

The present disclosure provides a culture medium and culture method for inducing olfactory mucosa mesenchymal stem cells into olfactory ensheathing cells. The culture medium of the present disclosure has a wide range of raw material source and clearly defined components. According to the culture method provided in the present disclosure, olfactory mucosa mesenchymal stem cells may be induced into olfactory ensheathing cells in stages. The method is simple, safe, and efficient. The induced cells express olfactory ensheathing cell markers GFAP and S100β, with a purity of 65% or more, thereby effectively addressing the source issue of olfactory ensheathing cells and exhibiting good application prospects in the field of nerve repair.

To further illustrate the present disclosure, the following detailed description is made by way of the examples and accompanying drawings below. The raw materials used in the following examples of the present disclosure are all commercially available.

### EXAMPLE 1 PREPARATION OF OLFACTORY ENSHEATHING CELL INDUCTION MEDIUM

The formulation included DMEM/F12 as a basal medium, and further included 0.2 mg/L transferrin, 0.5 μL/L insulin, 0.8 μL/L hydrocortisone, 0.4 μg/mL sodium selenate, 0.3 mL/L N-2 Supplement (100×), and 0.03 μg/L NT3.

### EXAMPLE 2 PREPARATION OF OLFACTORY MUCOSA MESENCHYMAL STEM CELLS

(1) The nasal mucosa donor signed an informed consent form, excluding infectious diseases such as syphilis, AIDS, and respiratory viruses, and intranasally administered with chloramphenicol eye drops for 3 days, 3 times daily, 3 drops each time.
(2) After nasal cavity disinfection and anesthesia, the superior turbinate and the upper lateral mucosa of the middle turbinate were excised using ethmoid sinus biting forceps, and the collected human olfactory mucosa sample was washed, where the washing preferably included washing the collected human olfactory mucosa sample 3 times with physiological saline until the blood stains were removed.
(3) The washed human olfactory mucosa sample was minced into tissue pieces smaller than 1 mm³ in size using sterile ophthalmic scissors.
(4) The tissue pieces were completely mixed with a mesenchymal stem cell culture medium, inoculated in a cell culture flask, followed by culture at 37° C. in a 5% CO₂ incubator, and after regular medium replacement and passaging, the olfactory mucosa mesenchymal stem cells were obtained.

The mesenchymal stem cell culture medium consisted of DMEM/F12 and 10% (v/v) fetal bovine serum.

### EXAMPLE 3 PREPARATION OF OLFACTORY ENSHEATHING CELL AND DETECTION

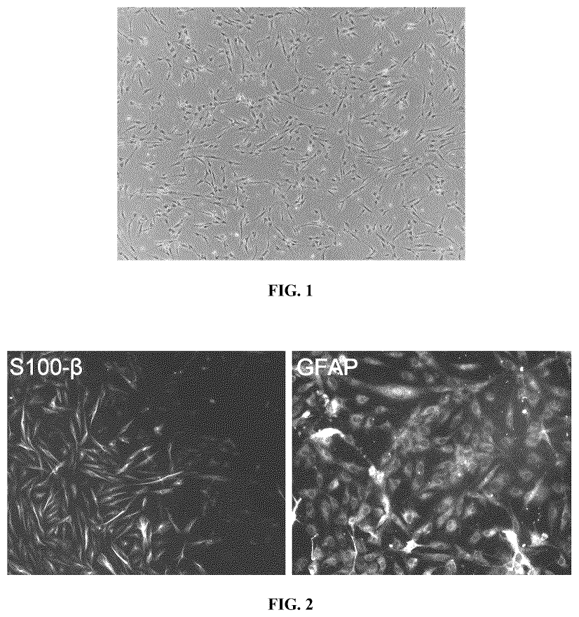

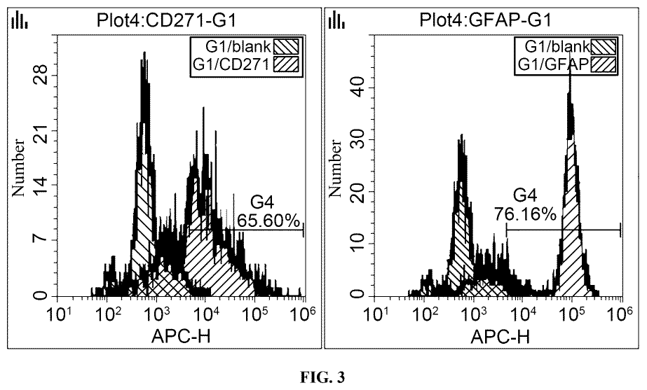

Specific steps were as follows:
(1) The obtained olfactory mucosa mesenchymal stem cells were cultured to P2, with 70-80% cell confluence.
(2) The above cells were digested with 0.25% (w/v) trypsin for 30 seconds, and centrifugation was performed at a rotation speed of 1000 rpm for 5 minutes.
(3) The supernatant was removed from the centrifuge tube, and the pellet was collected.
(4) The cell pellet was uniformly mixed with an olfactory ensheathing cell culture medium at a density of 10⁵ cells/ml.
(5) The above mixed solution was uniformly inoculated into a 6-well plate at 2 mL/well, and culture was performed at 37° C. in a humidified CO₂ incubator.
(6) After 1 day of culture, the supernatant and the floating cells were removed, and the olfactory ensheathing cell culture medium was replenished.
(7) Medium replacement was then performed every two days, with half-volume medium replacement for each time, and continuous culture was performed for 14 days to obtain the olfactory ensheathing cells, as shown in FIG. 1.
(8) The cells were collected, and the olfactory ensheathing cell markers were detected using immunofluorescence, as shown in FIG. 2.
(9) The cells were collected, and the olfactory ensheathing cell markers were detected using flow cytometry, as shown in FIG. 3, with a purity of 65% or more.

Further notes: By means of extensive long-term experimental exploration, the optimal formulation of the olfactory ensheathing cell induction medium was determined as: 0.1 to 0.5 mg/L transferrin, 0.5 to 1.0 μL/L insulin, 0.5 to 1.0 μL/L hydrocortisone, 0.1 to 0.5 μg/ml sodium selenate, 0.1 to 0.5 mL/L N-2 Supplement, and 0.01 to 0.05 μg/L NT3. The effects described in Example 3 can be achieved within these ranges.

The foregoing descriptions of the disclosed examples enable those skilled in the art to implement or use the present disclosure. Various modifications to the embodiments will be readily apparent to those skilled in the art, and the generic principles defined herein may be implemented in other embodiments without departing from the spirit or scope of the present disclosure. Therefore, the present disclosure will not be limited to the embodiments described herein but is in accordance with the widest scope consistent with the principles and novel features disclosed herein.

What is claimed is:

1. A cell culture medium for inducing olfactory mucosa mesenchymal stem cells into olfactory ensheathing cells, wherein the cell culture medium comprises DMEM/F12 as a basal medium, and further comprises transferrin, insulin, hydrocortisone, sodium selenate, N-2 Supplement, and Neurotrophin-3(NT3).

2. The cell culture medium of claim 1, wherein the cell culture medium comprises DMEM/F12 as a basal medium, and further comprises 0.1 to 0.5 mg/L transferrin, 0.5 to 1.0 μL/L insulin, 0.5 to 1.0 μL/L hydrocortisone, 0.1 to 0.5 μg/ml sodium selenate, 0.1 to 0.5 mL/L N-2 Supplement, and 0.01 to 0.05 μg/L NT3.

3. The cell culture medium of claim 1, wherein the cell culture medium comprises DMEM/F12 as a basal medium, and further comprises 0.2 mg/L transferrin, 0.5 μL/L insulin, 0.8 μL/L hydrocortisone, 0.4 μg/mL sodium selenate, 0.3 mL/L N-2 Supplement, and 0.03 μg/L NT3.

4. A method for inducing olfactory mucosa mesenchymal stem cells into olfactory ensheathing cells, comprising culturing olfactory mucosa mesenchymal stem cells using the cell culture medium of claim 1.

5. The method of claim 4, comprising the steps of:
(1) culturing olfactory mucosa mesenchymal stem cells to passage 2 (P2);
(2) digesting the cells using trypsin, and performing centrifugation;
(3) removing the supernatant from the centrifuge tube, and collecting the pellet;
(4) uniformly mixing the cell pellet with the cell culture medium to obtain a mixed solution;
(5) uniformly inoculating the mixed solution into a cell plate, and placing the plate in an incubator for culture;
(6) after 1 day of culture, removing the supernatant and the floating cells, and replenishing the cell culture medium, followed by regular medium replacement; and performing continuous culture for 14 days to obtain the olfactory ensheathing cells.

6. The method of claim 5, wherein the cell plate is a 6-well plate, with an inoculating volume of 2 mL/well.

7. The method of claim 5, wherein in step (2), the concentration of the trypsin is 0.25 w/v%, the digesting time is 30 seconds, the rotation speed of the centrifugation is 1000 rpm, and the centrifugation time is 5 minutes.

8. The method of claim 5, wherein in step (4), the inoculating density of cells in the cell culture medium is 10⁵ cells/mL.

9. The method of claim 5, wherein in step (1), the olfactory mucosa mesenchymal stem cells are cultured using an olfactory mucosa mesenchymal stem cell culture medium consisting of DMEM/F12 and 10 v/v% fetal bovine serum until reaching 70-80% cell confluence to obtain P2.

10. An olfactory ensheathing cell solution, wherein the olfactory ensheathing cell solution is prepared by the method of claim 4, and the olfactory ensheathing cells express olfactory ensheathing cell markers GFAP and S100β.

11. The method of claim 4, wherein the cell culture medium comprises DMEM/F12 as a basal medium, and further comprises 0.1 to 0.5 mg/L transferrin, 0.5 to 1.0 μL/L insulin, 0.5 to 1.0 μL/L hydrocortisone, 0.1 to 0.5 μg/ml sodium selenate, 0.1 to 0.5 mL/L N-2 Supplement, and 0.01 to 0.05 μg/L NT3.

12. The method of claim 4, wherein the cell culture medium comprises DMEM/F12 as a basal medium, and further comprises 0.2 mg/L transferrin, 0.5 μL/L insulin, 0.8 μL/L hydrocortisone, 0.4 μg/mL sodium selenate, 0.3 mL/L N-2 Supplement, and 0.03 μg/L NT3.

13. The method of claim 5, wherein the cell culture medium comprises DMEM/F12 as a basal medium, and further comprises 0.1 to 0.5 mg/L transferrin, 0.5 to 1.0 μL/L insulin, 0.5 to 1.0 μL/L hydrocortisone, 0.1 to 0.5 μg/ml sodium selenate, 0.1 to 0.5 mL/L N-2 Supplement, and 0.01 to 0.05 μg/L NT3.

14. The method of claim 5, wherein the cell culture medium comprises DMEM/F12 as a basal medium, and further comprises 0.2 mg/L transferrin, 0.5 μL/L insulin, 0.8 μL/L hydrocortisone, 0.4 μg/mL sodium selenate, 0.3 mL/L N-2 Supplement, and 0.03 μg/L NT3.
